(19) United States
(12) Patent Application Publication
(10) Pub. No.: US 2015/0025506 A1
(43) Pub. Date: Jan. 22, 2015

(54) APPARATUS AND METHOD FOR FLUID DELIVERY

(71) Applicant: FormationD Pty Ltd, Sydney (AU)

(72) Inventor: Eric Chau, Botany (AU)

(73) Assignee: FormationD Pty Ltd, Sydney (AU)

(21) Appl. No.: 14/346,235

(22) PCT Filed: Sep. 21, 2011
(86) PCT No.: PCT/AU2011/001219

§ 371 Date: Sep. 23, 2014

(51) Int. Cl. A61F 9/00

(52) U.S. Cl. CPC A61F 9/0026
USPC 604/521; 604/301

(57) ABSTRACT

The present invention relates to an apparatus and method for delivering a fluid, and particularly, but not exclusively, to an apparatus and method for delivering a fluid to an eye. The apparatus provides a useful device that assists users to accurately deliver fluid to the eye and includes features that make using the apparatus more comfortable.

### FIELD OF THE INVENTION

The present invention relates to an apparatus and method for delivering a fluid, and particularly, but not exclusively, to an apparatus and method for delivering a fluid to an eye.

### BACKGROUND OF THE INVENTION

Conventional eye droppers are generally in the form of squeezable container with a restricted fluid outlet. The container may usually be made of plastics and is arranged to be squeezed by a person's fingers. The restricted fluid outlet is formed by an elongate substantially conical applicator with the fluid outlet at the tip of the cone. The applicator is provided with a cover, such as a screw on cap. Medication is contained in the plastics container. In use, the cap is removed and the eye dropper is manoeuvred into position such that the applicator tip is immediately above the eye so that the fluid can be delivered into the eye. Once the user has positioned the applicator in the correct position the user squeezes the plastic container to force a one or more drops of medication out of the applicator and into the eye.

Other eye droppers are in the form of a substantially rigid container with a restricted fluid outlet. The container may be made from rigid plastics and is arranged to deliver fluid by gravity. In use these rigid eye droppers are turned so that the outlet is positioned above the eye and the fluid within the containers flows out of the container due to gravity. These types of eye droppers require the user to correctly aim and correctly position the eye dropper outlet above the eye in order to deliver fluid to the eye. Further delivery due to gravity can be time consuming and difficult to control.

The eye is a relatively small area and difficult to provide medication to. This difficulty is compounded by the fact that the eye is one of the most sensitive areas of the human body. The natural reflex is to close the eye when it gets any foreign object (for example a droplet or two of medication) coming towards it. It is also difficult to align the applicator perfectly in line with the eye. Often, with conventional eye droppers, the medication falls on the users face or the eyebrow or on the eyelashes because the user closes his or her eye as the drop approaches the eye. A further difficulty is that the hand and/or arm need to be extended and bent into an awkward position to accurately apply the medication to the eye. A user with joint or muscular disabilities may not have the dexterity required to apply eye drops. Elderly users, for example, may not have a sufficiently steady hand. Application of the medication to the eye with a conventional eye dropper can be a difficult and frustrating process.

Conventional eye droppers also generally include a screw on cap. Screw on caps can be difficult to remove and may require excessive force for removal. It can often be difficult for an elderly person or a person with weakness in their arms and hands to securely close or open a screw cap. A further problem associated with eye droppers is that the applicator tip is exposed and may contact the fingers of a user, or face, or other potentially contaminating surface this may lead to contamination of the medication.

### SUMMARY OF THE INVENTION

According to a first aspect, the present invention provides a closure apparatus for a device for delivering fluid to an eye, the closure apparatus comprising a lid arranged to be moved from a first position, in which the lid closes a fluid delivery opening of the device, to a second position in which the fluid delivery opening of the device is open to enable dispensation of fluid, the closure apparatus having an eye rest projecting outwardly from a side wall of the lid that facilitates opening of the fluid delivery opening, the eye rest being arranged to rest against the eye surrounding anatomy of a user when the lid is in the second position during the dispensation of fluid in order to steady the device for delivery of fluid.

In an embodiment the closure apparatus comprises a base that is arranged to engage with a fluid container, the lid of the closure apparatus being arranged to mate with the base to close the fluid delivery opening.

In an embodiment the lid is coupled the base by a hinge, the hinge being disposed generally opposite the eye rest and connecting to a side wall of the base.

In an embodiment the eye rest bears a message.

In an embodiment the eye rest comprises a contoured surface that is arranged to conform to the eye surrounding anatomy of a user such that the eye rest bears against the eye surrounding anatomy of a user to allow for greater stability of the apparatus when in use.

In an embodiment the closure apparatus comprises a depression within the lid, the depression shaped to receive a finger of a user to allow a user to grasp the closure apparatus.

In an embodiment the hinge is arranged to facilitate movement of the lid from the first position to the second position, and lock the lid in the second position when the lid position exceeds a predefined locking angle.

In an embodiment, the closure apparatus may be utilized with a container for delivering fluid. For example, it may be utilized to close a container to arranged deliver fluid to the eye such as a medicament.

In accordance with a second aspect of the present invention there is provided an apparatus comprising a nozzle for the dispensation of fluid from the fluid delivery opening of the device and a collar disposed concentrically about the nozzle, the nozzle being seated within the collar with the fluid delivery opening disposed below the top of the collar, whereby to protect the nozzle.

In an embodiment the collar has at least one slot, the at least one slot being arranged to guide fluid out of the collar.

In an embodiment the collar comprises an upper edge with a projecting lip, the lip being arranged to break the surface tension of any fluid about the collar so as to reduce the chance of fluid flowing back into the nozzle opening.

In an embodiment the apparatus comprises a reflective surface disposed about the nozzle, the reflective surface in use enabling a user to view alignment of the nozzle and the fluid delivery opening with their eye and accurately deliver fluid to the desired area.

In accordance with a third aspect of the present invention there is provided an apparatus for facilitating delivery of fluid, the apparatus comprising a nozzle for dispensing fluid from the fluid delivery opening of the device, the nozzle including an arrangement for breaking surface tension of a droplet being delivered at the outlet, whereby to reduce the flow of fluid back into the bottle when the droplet is dispensed.

In an embodiment the arrangement comprises a step in the wall surrounding the orifice, the step arranged to break the surface tension.

In an embodiment the apparatus further comprising a collar disposed about the nozzle.

In a further embodiment the collar comprises a lip for breaking surface tension of a droplet being delivered at the outlet, whereby to reduce the flow of fluid back into the nozzle when the droplet is dispensed.

In an embodiment the eye rest is rigidly connected to the side wall of the lid.

In an embodiment the eye rest is integrally formed with the lid.

In an embodiment the eye rest extends substantially perpendicular to the side wall of the lid.

In an embodiment the eye rest functions as a lever to facilitate opening of the fluid delivery opening.

In accordance with a fourth aspect of the present invention there is provided an apparatus for facilitating delivery of a fluid comprising a translucent or transparent container arranged to contain the fluid and a closure apparatus in accordance with any one of the preceding claims.

In accordance with a fifth aspect of the present invention there is provided a method of delivering fluid to a user's eye, using an apparatus for facilitating the delivery of fluid, comprising the steps of:
opening a lid of the apparatus, the lid having an eye rest projecting outwardly from a side wall that facilitates opening,
positioning the eye rest on or about eye surrounding anatomy such that the rest bears against at least part of the eye surrounding anatomy
aligning an outlet of the apparatus with the user's eye and
providing fluid to the user's eye via the outlet.

In a further embodiment the step of aligning the outlet comprises the additional step of viewing a reflection of the user's eye within a reflective surface disposed on an upper surface of the apparatus.

In an embodiment the fluid could be a fluid medicament.

In accordance with a sixth aspect of the present invention there is provided an eye drop dispenser comprising:
a base that engages with a fluid container, the base having a nozzle that defines a fluid opening for dispensing eye drops from the fluid container,
a lid that mates with the base to close the fluid opening, the lid having a cap that is generally commensurate with the fluid opening of the nozzle,
a hinge that couples the lid to the base so that the cap blocks the fluid opening of the nozzle when the lid is in a closed position, the hinge connecting to a side wall of the base, and
an eye rest that extends outwardly from a side wall of the lid to rest against a user's eye surrounding anatomy during dispensation of eye drops, the eye rest being disposed generally opposite the hinge to facilitate opening of the lid.

In an embodiment the dispenser comprises a reflective surface disposed about the nozzle that aids alignment of the nozzle with a user's eye.

In an embodiment the dispenser comprises a collar disposed about the nozzle that shields the nozzle from contamination, the fluid opening being disposed below an upper lip of the collar.

In an embodiment the collar includes a slot that defines an opening for the passage of eye drops.

In an embodiment the dispenser comprises a step formed within the nozzle adjacent the fluid opening that reduces fluid surface tension during sys drop dispensation.

In an embodiment the eye rest is rigidly connected to the side wall of the lid.

In an embodiment the eye rest is integrally formed with the lid.

In an embodiment the eye rest extends substantially perpendicular to the side wall of the lid.

In an embodiment the eye rest functions as a lever to facilitate opening of the fluid delivery opening.

In an embodiment the eye rest bears a message.

### BRIEF DESCRIPTION OF FIGURES

Features and advantages of the present invention will become apparent from the following description of embodiments thereof, by way of example only, with reference to the accompanying drawings, in which,

FIG. 1 shows a system for delivering fluid, in particular a system for delivering fluid to the eye of a user, in accordance with an embodiment of the present invention;

FIG. 2 shows a perspective view of the system of FIG. 1, with an open lid;

FIG. 3 shows a perspective view from underneath of an apparatus for facilitating the delivery of fluid medicament, in accordance with an embodiment of the present invention;

FIG. 4 shows another perspective view from above of the apparatus of FIG. 3;

FIG. 5 shows a further perspective view from above of the apparatus of FIG. 3;

FIG. 6 shows a cross-sectional view of the apparatus of FIG. 3;

FIGS. 7 and 8 show views of a system in accordance with an embodiment of the invention in use.

### DETAILED DESCRIPTION

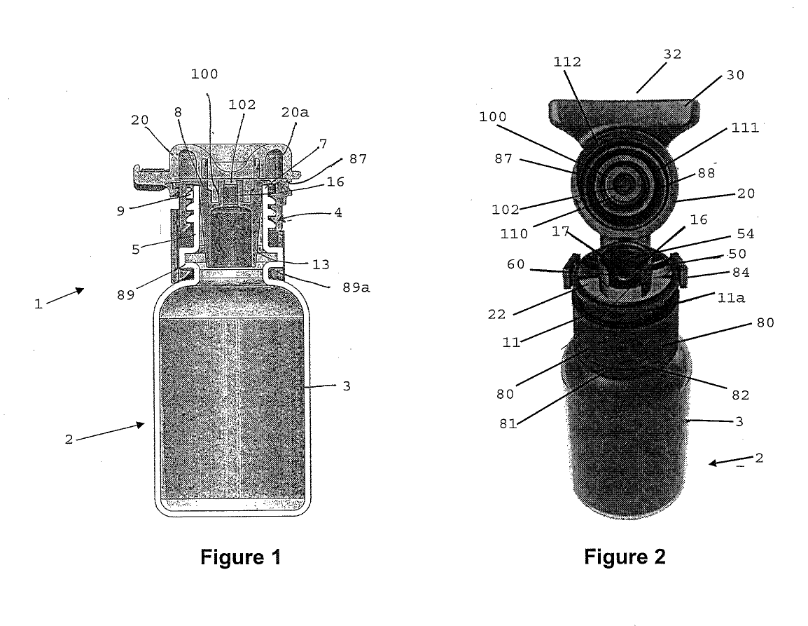

FIG. 1 shows a system 1 for delivering fluid. In particular the system 1 is arranged for delivering fluid to the eye of a user.

The system for delivering fluid comprises a container 2. The container 2 includes a hollow body 3 arranged to hold fluid within it. The fluid is any suitable eye medicament fluid or any other fluid suitable for use with a human eye for example rinse eye drops or steroid eye drops, dry eye treatment drops or any other such eye drops. In the illustrated embodiment the container 2 is in the form of a bottle. In other embodiments the container can be any other suitable shape such as a vial, an ampoule, a rectangular container or any other suitable container shape.

The body 3 may be resilient and substantially flexible such that the body 3 flexes when squeezed by a user, to expel the contents of the body 3 and the container 2. The body 3 returns to its original shape after being squeezed.

The container 2 may be formed from any suitable plastics material that allows the body 3 and the container 2 to be substantially flexible, for example polyethylene or polypropylene or any other suitable plastics material. In an alternative form the body 3 of the container 2 may be substantially rigid and may not flex when squeezed by a user. In this alternate form the contents of the body 3 may be expelled due to gravity, by the user turning the container 2 “upside down” to allow the contents of the container 2 to flow out due to gravity.

The container 2 comprises an outlet 4. The outlet 4 includes a neck 5 that extends outwardly from the body 3. The neck 5 is preferably hollow and defines an outlet passage 6. In one embodiment the neck 5 is substantially cylindrical in shape. In other embodiments the neck 5 may be a rectangular shape, tubular shape, or any other suitable shape. The neck 5 includes a distal end 7 and an outlet opening 8 is formed in the distal end 7. The outlet 4 further includes a thread 9 or a plurality of thread. The thread 9 is formed on the neck 5 and extends about the neck 5. The thread 9 can be any suitable pitch. In one example the thread may have a pitch of 2 mm, but alternatively the thread can have a pitch of between 0.5 mm-10 mm depending on the size of the container 2 and the neck 5. The thread 9 may have any other appropriate pitch size. The thread 9 may have any suitable pitch angle.

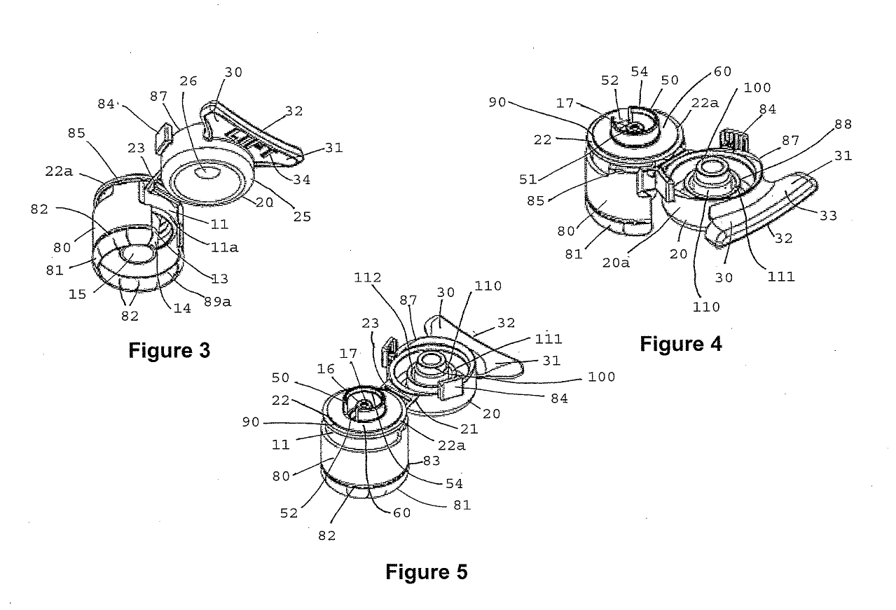

The system 1 further comprises an apparatus 10 for delivering fluid medicament. The apparatus 10 is illustrated in detail in FIGS. 3 to 6. In one form the apparatus 10 is arranged to be removably attached to or about the outlet 4 of the container 2. As shown in FIGS. 1 and 2, the apparatus 10 is positioned about the outlet 4 of the container. The apparatus 10 may be a cap that is arranged to be positioned on or about the container 2 or any other such fluid dispensing container, for example eye droppers, ear droppers and the like. The apparatus 10 comprises several features and advantages which will be described below.

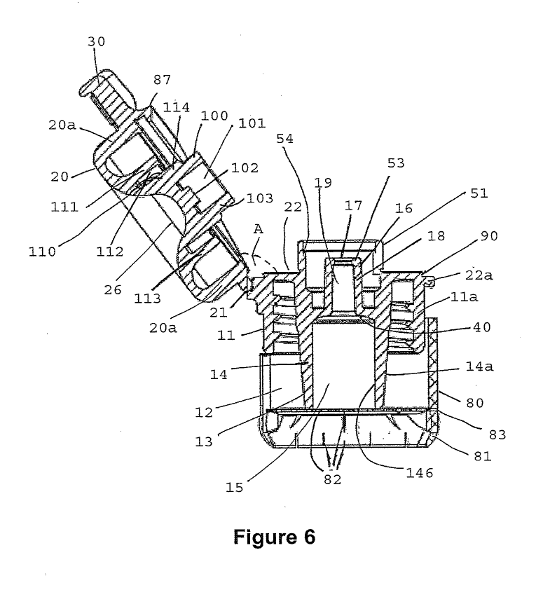

FIGS. 3 to 6 show detailed views of an apparatus for facilitating the delivery of fluid to a user's eye. FIGS. 3 to 5 are perspective views of the apparatus 10 and its various parts. FIG. 6 is a cross-sectional of the apparatus 10.

The apparatus 10 for delivering fluid medicament is in the form of a cap and comprises a body 11. The body 11 comprises a continuous wall 11a. The body 11 comprises a skirt 80, which will be described in more detail later on. The wall 11a and skirt 80 are substantially cylindrical in shape. The wall 11a or skirt 80 or both define a hollow region 12. In alternative embodiments the body 11 and wall 11a and skirt 80 can be any other suitable shape. The apparatus further includes a projecting portion 13 that extends within the outer wall 11a of the body 11, within the hollow region 12. The projecting portion 13 comprises wall 14. The wall 14 may be one continuous wall that is cylindrical in shape. The outer surface 14a of the wall 14 tapers inwardly. The inner surface 14b of the wall 14 does not taper and extends substantially vertically. The projecting portion 13 in this embodiment may be a frustro-conical shape. In other embodiments the projection may be rectangular shaped or any other shape. Alternatively the projecting portion 13 may comprise a plurality of walls that taper inwardly the projecting portion 13 includes a passage 15. The walls 14 of the projecting portion 13 define the passage 15. The passage

The apparatus 10 is arranged to be positioned about the outlet 4 of the container 2, in use. When in use, the outlet 4 extends into the hollow space 12 of the apparatus 10. The wall 11a, of the body 11 is positioned around the outside of the neck 5 of the container 2. The projection 13 is arranged to be inserted into the passage 6 defined by the neck 5. The projection 13 further includes a nozzle 16 extending outwardly from the body 11 opposite the direction of the projecting portion 13. The nozzle 16 includes an outlet opening 17 and the nozzle wall 18 defining a nozzle passage 19. The body 11 of apparatus 10 comprises a top wall 22a, that has an upper surface 22.

The apparatus further comprises a closure apparatus that includes a lid 20. The lid 20 may be flexible. The lid 20 includes a continuous cylindrical wall 20a. Alternatively the lid may comprise a plurality of walls 20a, the walls being parallel to each other. In the illustrated embodiment wall 20a is substantially cylindrical. The wall 20a defines a lid body 25. The lid 20 is attached to the body 11 by a hinge 21 and can be moved from an open position to a closed position. FIG. 1 shows the lid 20 in a closed position and FIG. 3 shows the lid 20 in an open position. The lid wall 20a has a continuous edge 87. In the closed position, part of the lid 20, in particular the edge 87 rests on the upper surface 22 of the body 11.

The hinge 21 can be any suitable hinge that connects the lid 20 to the body 11 of the apparatus 10 allowing the lid 20 to be rotated about the hinge 21. In this embodiment the hinge 21 is a locking “living hinge” that locks the lid 20 in an open position. In one embodiment the hinge 21 may include a web 23. The web 23 may be substantially thin and flexible. The web 23 and hence the hinge 21 may be substantially resilient. The hinge 21 may be formed integral with the body 11. In one form the locking living hinge 21 may be formed as part of the apparatus body 11 and may be formed when the apparatus for fluid delivery 10 is formed. The hinge 21 has a locking angle A, such that once the lid 20 is opened past the locking angle A, the lid 20 remains locked in the open position. If the lid 20 is in a position below the locking angle A, the hinge forces the lid 20 to a closed position because of the biasing forces in the hinge 21, in particular the web 23. The web 23 acts as a biasing member, biasing the hinge 21 and hence the lid 20 to a closed position if the lid 20 is at a position that is less than the locking angle A. If the lid 20 is in a position where it exceeds the locking angle A, then the web 23 biases the lid 20 to an open position. These biasing forces are created due to the web 23 being resilient and in one form the fibres in the web are aligned with each other when the hinge 21 is formed. The locking angle A is greater than 90 degrees and in one form can between 95 degrees and 150 degrees relative to the horizontal.

The lid 20 remains locked in the open position due to the material properties of the web 23 when the hinge is manufactured. If the lid 20 is in a position that is less than the locking angle A, the lid 20 is closed due to the biasing forces of the fibres of the web 23.

In an alternate form the hinge 14 may be a separate part that is attached to the body 11 of the apparatus by any suitable fastener or mechanism, for example by plastic welding, bolting, gluing or any other such process.

The hinge 23 is advantageous because it allows the lid 20 to be locked into an open position. This allows the user to use the apparatus without having the lid 20 close during use. The hinge 21 is also advantageous because of the web 23. The web 23 is flexible and hence allows the lid 20 to be easily operated by a weaker person for example, an old person, a child, or a person with weakness in the arms and/or hands.

A further advantage of the hinge 21 is that it ensures the lid 20 remains attached to the apparatus 10 and cannot be lost (as may be the case with eye droppers in the prior art). Further since the lid 20 does not need to be removed there is a reduced chance of contamination of the lid 20 because it does not need to be handled and does not need to be placed on a potentially contaminated surface, such as a table top. This makes the apparatus 10 safer to use since there is a reduced chance of contamination.

The lid 20 is provided with a rest 30, which is arranged to rest against eye surrounding anatomy and steady the apparatus 10 in use. In this embodiment, the rest 30 comprises a projecting tab 31 which is arranged to rest on the anatomy underneath the eye or on the eyebrow or on the anatomy adjacent the eye. The rest 30 may be formed integrally with the lid 20. In the illustrated embodiment the rest 30 extends in an opposite direction to the hinge 21.

Alternatively the rest 30 may be one or more projections (not illustrated) that extend from the lid 20. The projections may be spaced equally about the lid 20. The projections may be integrally formed with the lid 20. The projections may be in the form of prongs or extensions of any suitable shape that are arranged to rest against eye surrounding anatomy when in use, to stabilize the apparatus 10 and/or system 1 to accurately deliver fluid to a user's eye.

In an alternative form the rest 30 may be formed as a separate piece or pieces and can be attached to the body 11 of the apparatus 10. In this alternate embodiment the rest 30 may be a projecting tab that extends from a portion of the body 11. As a further alternative the rest may be one or more projections that extend outward from the body 11. The rest 30 may extend from the upper surface 22 of the apparatus 10. The rest 30 is arranged to bear against the eye surrounding anatomy and stabilise the apparatus 10 when in use.

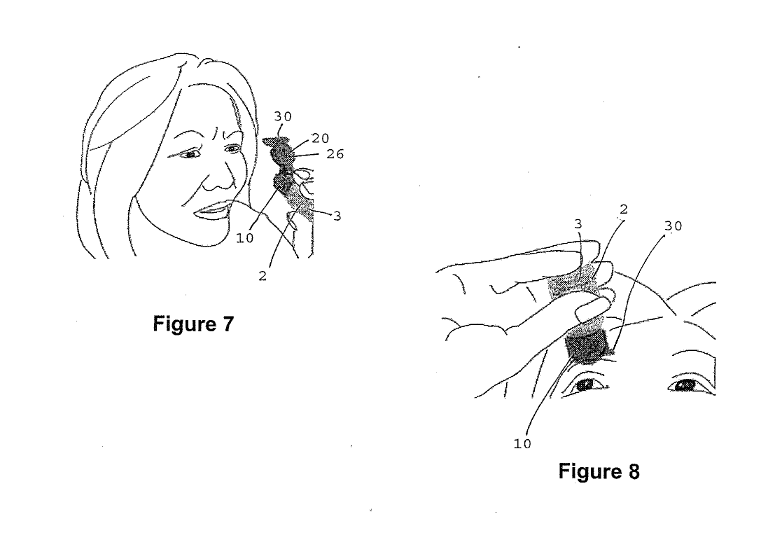

The rest 30 is advantageous because it provides stability of the apparatus for fluid delivery when in use. The rest 30 is also advantageous because when the rest 30 is positioned on eye surrounding anatomy, the outlet opening 17 and the nozzle 16 and outlet 6 of the container 2 are substantially aligned with the eye of the user to allow the fluid to be delivered to the eye accurately. In one embodiment the rest 30 is arranged to rest on or adjacent the eyebrow 100 of a user, as shown in FIG. 7.

The rest 30 comprises a contoured resting surface 32 that may conform to the shape of eye surrounding anatomy of a person. In one form the contoured surface 32 is curved such that it can conform to a the eye surrounding anatomy such as at least part of the orbital bone, to allow the rest 30 to bear against the eye surrounding anatomy to allow for greater stability of the apparatus 10 when in use. The rest is arranged to sit against the user's eye surrounding anatomy to reduce error due to the shaky hands. The rest 30 is advantageous for use by old people or people with shaky hands because it provides stability and alignment of the outlet opening 17 of the nozzle 16 with the eye of the user. This alignment provides better dosage compliance because there is a reduced chance of the fluid landing outside the eye, on the cheek or eyebrow for example. The rest 30 allows the apparatus 10 to be more dosage compliant because there is a greater chance that the fluid will be delivered to the eye of the user, because the apparatus is stabilised when in use and the outlet 17 of the nozzle 16 is aligned with the user's eye.

The projecting tab 31 additionally allows for easy lifting of the lid 20. The tab 31 is shaped so that the lid 20 can be lifted by a finger or by using an edge of a piece of furniture for example a table. The tab 31 includes a large flat area 33 to allow finger to lift the lid 20. The tab 31 is advantageous because the lid 20 can be easily lifted to an open position. Many traditional eyedroppers use a screw top lid that need to be screwed off. This can often require too much force or dexterity that older people or weaker people can find difficult to open. The tab 31 allows for easy opening of the lid 20 by older people or people with weakness in their arms /hands.

The tab 31 may include an embossed portion 34, as shown in FIG. 4. The embossed portion 34 may include any suitable message that explains to the user how to use the eyedropper. In the illustrated example the embossed portion says LIFT, which directs a user to lift the tab 31 to open the lid 20. The embossed portion 34 is advantageous because it makes the eyedropper easier to use due to the visual cues that can be provided on the tab 31.

The lid 20 comprises a body 25. The body 25 includes a depression 26. The depression 26 is substantially circular and conical shaped. The depression 26 is shaped to receive a finger for example an index finger when a user may be opening the lid 20. The depression 26 is advantageous because it is shaped to accommodate a finger and allows for an extra grasping point on the lid 20. The depression 26 can make opening the lid 20 easier. The depression 26 also allows a person to grasp the apparatus 10 and system 1 easily and comfortably.

As discussed above the projection 13 of the body 11 defines a passage 15 that extends within the projection 13. In one embodiment the passage 15 is substantially cylindrical due to the cylindrical walls 14 of the projection. The passage 15 allows delivery of fluid from the passage 15 to the outlet 17 of the apparatus. The nozzle 16 extends from the projection and is narrower than the projection 13 and is narrower than the passage 15.

The nozzle 16 defines a nozzle passage 19. The passage 15 and nozzle passage 19 are in fluid communication. The passage 15 and the nozzle passage 19 share a common vertical axis so as to allow fluid from the container to pass through the passage 15, through the nozzle passage 19 and out of the outlet 17. The projection 13 has a greater diameter than the nozzle 16, and the passage 15 has a greater diameter than the nozzle passage 19. The projection 13 includes a throat section 40. The throat section 40 is an area of reduced internal diameter within the projection 13. The nozzle 16 extends from the throat 40. The throat 40 may include a chamfered section 41 that reduces the internal diameter of passage 15, as illustrated in FIG. 6. The throat section 40 having a reduced internal diameter reduces the amount of fluid entering the nozzle 16. This is advantageous because the throat section 40 reduces the possibility of excessive fluid being delivered to a user's eye. The nozzle 16 has smaller diameter than the projection 13. The nozzle passage 19 having a smaller diameter than the passage 15 is advantageous because it reduces the amount of fluid entering the nozzle 16, thus reducing the possibility of excessive fluid being delivered to a user's eye.

The apparatus 10 further comprises a collar 50 that extends outwardly from the upper surface 22 of the apparatus 10. In the illustrated embodiment the collar 50 extends at least partially around the nozzle 16. In another form the collar 50 may extend completely around the nozzle 16. The height of the collar 50 is greater than the height of the nozzle 16, such that the top edge 51 of the collar 50 is higher than the outlet 17 of the nozzle 16.

In an alternative embodiment, the collar 50 may be in the form of a plurality of raised extensions that extend from various locations on the upper surface 22. In one embodiment the extensions may not completely surround the nozzle. In one embodiment the collar 50 may surround the nozzle 16.

There may one or more cut out sections 52 in the collar 50. The one or more cut outs 52 may be any suitable shape such as a V shape, a rectangular shape, a circular shape, an elliptical shape and so on. In the illustrated form of the cut out is rectangular shaped and allows for the fluid to be easily dispensed. In the illustrated embodiment the collar includes one cut out 52, to expose the nozzle 16 and the outlet 17. The cut-out 52 can further facilitate the delivery of fluid from the outlet. The cut-out 52 can serve as a guide, to guide the fluid out of the outlet 17, when in use.

The collar 50 extends higher than the nozzle 16 and the outlet 17 to protect from a user touching nozzle 16 when using the apparatus 10 or system 1. This is advantageous because it reduces the chances of contaminating the nozzle 16 by a user touching the nozzle 16. In some traditional eyedroppers the conical applicator is exposed and a user often touches the applicator, which can lead to contamination. The collar 50 is advantageous because it increase the chances of compliance with safety standards because of the reduced chance of contamination of the nozzle 16 or the fluid as it comes out the outlet 17.

In one embodiment the nozzle 16 may include a step 53 that is formed within the nozzle wall 18. The step 53 is adjacent the outlet 17. The step 53 breaks the surface tension of a drop formed at the outlet 17 by allowing any water that hits the step 53 to channel away from the outlet 17. The step 53 in the nozzle 16 reduces the possibility of a drop falling back into the nozzle 16 and the container 2. This reduces the possibility of contamination because drops do not fall back into the nozzle 16 and container 2. In an alternative embodiment the collar 50 includes a lip 54. The lip 54 may be disposed on the upper edge 51. The lip 54 may break the surface tension of a drop at or near the outlet 17 or a drop formed in the collar 50 by allowing any water that impinges on the lip 54 to channel away from the outlet 17. The water can be channel out through the cut out 52. This reduces the chances of a drop falling back into the nozzle 16 and container 2 and reduces the possibility of contaminating the medical fluid in the container 2.

The upper edge 51 of the collar 50 is of a rounded form. When viewed from the side, the collar 50 has a flat profile. The upper surface 22 and collar 50 do not have a pointed projection unlike traditional eyedroppers which include a pointed cone or a pointed tip. The nozzle 16 also may have a flat profile. The collar 50, the rounded edge 51 of the collar 50, the flat profile of the collar 50 and the flat profile of the nozzle 16 make the apparatus 10 less intimidating to use. People are often afraid of getting poked in the eye by the tips of many traditional eyedroppers. Further the rest 30 stabilises the eyedropper when in use which also adds to the feeling of safety and security when using the apparatus 10 and system 1.

The apparatus 10 also includes a reflective surface 60 disposed on the upper surface 22. The reflective surface 60 is substantially planar and flat with minimal blemishes to provide optimal reflectivity. The reflective surface 60 may be formed any suitable reflective material such as tin, aluminium, silver, or glass with a coating or tin, copper. The reflective surface 60 is preferably formed from a thin piece of reflective material such as a film with a reflective side that is disposed on the upper surface 22. In one embodiment the reflective surface 60 may be formed from reflective ink that is disposed on the upper surface 22. The reflective surface 60 can be positioned on the upper surface 22 by any suitable process such as deposition, powder deposition, gluing, stamping or painting. The reflective surface 60 is advantageous because it allows the user to focus on the nozzle 16 or the outlet 17 when the rest 30 is positioned on the eye surrounding anatomy. The reflective surface 60 allows a user to accurately align the nozzle outlet 17 with the user's eye by looking at the reflection of their eye in the reflective surface 60.

The apparatus 10 further comprises a thread 70 formed on the inside of the wall 11a of the body 11. The thread 70 of the apparatus 10 is complementary to the thread 9 of the container 2. The thread 70 allows the apparatus to be fitted onto to existing containers 1 or eyedropper bottles, or contains specially made o fit to the apparatus. In an alternative embodiment the apparatus may be include a bayonet fitting to allow the apparatus 10 to be fitted over the outlet 4 of an existing bottle or container 2. The apparatus 10 may be positioned over the outlet 4 of a container 2 by a snap fit arrangement.

The apparatus 10 comprises a skirt 80 depending from surface 22 and surrounding the projection 13. The skirt 80 is flexible and resilient. The skirt 80 is resilient such that it can snap on to the outlet 4 of any container 2 or any eyedropper bottle. The skirt 80 may comprise a removable ring 81 that may be formed as part of the skirt 80. The ring 81 may include areas of weakness 82. The ring 81 may be a perforated section of the skirt 80. The ring 81 may be positioned along the bottom edge 83 of the skirt 80. The ring 81 is a tamper evident ring. The ring 81 is perforated to indicate tamper by a person. The ring 81 can be removed due to the perforations or areas of weakness 82. Tamper with the ring 80 or the apparatus 10 can be indicated if the ring 81 is removed.

The neck 5 of the container 2 comprises a projection 89 extending outward from the neck 5 and around the neck 5. The projection 89 may be a flange. The ring 81 comprises inwardly projecting tooth 89a. The tooth 89a extends about the ring 81. In another form the ring 81 may comprise plurality of projecting teeth 89a. The tooth 89a engages with the projection 89 to form a snap fit connection as shown in FIG. 1. As the body 11 is unscrewed from the neck 5, the ring 81 remains attached to the neck due to the tooth 89a. The ring 81 comes away from the skirt 80 because of the areas of weakness 82 and the ring may remain attached to the neck. The ring 81 remaining attached to the neck 5 shows that the apparatus 10 and system 1 have been used or tampered with. In another form the skirt 80 may come away from the body 11, as the body 11 is unscrewed from the neck 5. The skirt 80 may include further weakened areas (not shown) at the connection point between the skirt and the body 11, or upper wall 22a. If the skirt 80 and ring 81 are attached to the neck 5 and the body 11 is not, can be an indication of tampering or prior use.

The apparatus 10 further comprises one or more removable stubs 84. The stubs 84 are flexible and can be snapped off or removed from the body 11. The stubs 84 serve as tamper evident devices. When the apparatus 10 is formed, the stubs 84 are initially attached to both the lid 20 and the body 11. When the lid 20 is opened the stubs 84 can snap off the body and remain attached to the lid 20. In another embodiment when the lid 20 is opened the stubs 84 remain attached to the body 11 and snap off from the lid 20. Tampering of the container 2 or the apparatus 10 can be indicated if the stubs 84 are attached to only one of the lid 20 or the body 11. In a further embodiment the skirt 80 may include weakened sections 85 where the stubs 84 attach to the skirt 80. The weakened sections 85 allow the stubs 84 to be easily snap off and show tampering with the apparatus 10. The lid 20 may also include weakened sections 86 when the stubs 84 attach. The weakened areas 86 allow the stubs 84 to snap off and show tampering with the apparatus 10.

The lid 20 of the apparatus 10 includes an edge 87 that is arranged to rest against the upper surface 22 when the lid 20 is in a closed position. The upper surface 22 includes a raised portion 90 that extends upward from the upper surface 22. In use the edge 87 is dimensioned such that it fits around the raised section 90 to form an air tight seal when the cap is closed. The raised section 90 and the edge 87 form a snap fit closure, meaning the lid 20 snaps closed. The air tight seal is advantageous because it reduces the chance of contamination of the nozzle 16 when the apparatus is not in use.

The lid 20 further includes a hollow 88 area formed within the body 25 of the lid 20. The lid 20 comprises a raised flange 100 positioned within the hollow 88 area. The flange 100 includes an orifice 101. The orifice 101 is arranged to fit over the nozzle 16 when the lid 20 is snap closed. The flange 100 is arranged such that the orifice 101 and nozzle 16 are aligned when the lid 20 is moved to a closed position. The orifice 101 includes a plug 102 positioned within the orifice 101. The plug 102 is aligned with the nozzle outlet 17 and fits into the outlet 17 when the lid 20 is in a closed position to form a sealed closing. The plug 102 is positioned within the orifice 101 such that the walls 103 of the flange 100 are greater in height that the plug 102. The plug 102 cannot be touched by a user thus reducing the chance of contamination making the plug 102 and flange 100 arrangement safer and clear for use.

In one form the lid 20 may include, a first flange 100, a second flange 110 and a third flange 111 extending from the lid 20. The flanges 100, 110, 111 may all be co-axially aligned with each other and are concentric with each other. The third flange 111 is greater in diameter than the second flange 110, which is greater in diameter than the first flange 100. The second flange 110 is positioned within the third flange 111, and first flange 100 extends from the second flange 110. A space 112 is defined in between the wall 113 of the third flange 111 and the wall 114 of the second flange 110. The space 112 is arranged to receive the collar 50, in particular the edge 51 of the collar 50 to form an air tight seal between the third boss 111 and the collar 50. This air tight seal in addition to the seal between the raised section 90 and the edge 87 of the lid 20 provide additional protection against contamination of the medical fluid within the container.

The apparatus 10 or cap and all its parts may be moulded and formed as a single moulding. Any suitable one shot moulding process can be used to form the apparatus 10 as described above. In alternative embodiments the apparatus 10 and its parts may be vacuum formed or cast or injection moulded. Any other suitable forming process can be used. The apparatus 10 and its constituent parts are formed from a suitable plastics material such as polypropylene. In other embodiments the apparatus and its constituent parts may be made from materials such as rubber or silicone. In still further embodiments the apparatus 10 and some or all of its parts could be made from stainless steel or any other suitable metal that can be sterilised easily. The apparatus may be machined or cast if made from a metal.

In a further embodiment the container 2 and apparatus for fluid delivery 10 may be substantially translucent or transparent to allow light to pass through the container 2 and the apparatus for fluid delivery 10. This is advantageous because the light passing through the apparatus 10 and container 2 allows a user to more clearly view the outlet 17 and hence allow the user to accurately align the outlet 17 with his/her eye in use to allow for more accurate delivery of the medical fluid.

A method of delivering fluid medicament to a user's eye, using the apparatus 10 or system 1, for facilitating the delivery of fluid medicament as described earlier, comprises the steps of:
opening a lid of the apparatus,
positioning a rest of the apparatus on or about eye surrounding anatomy such that the rest bears against at least part of the eye surrounding anatomy,
aligning an outlet of the apparatus with the user's eye,
providing fluid medicament to the user's eye via the outlet.

The apparatus and system are the same as described earlier. FIG. 7 shows a user having opened the lid of the apparatus 10 and checking the reflection in the reflective surface 60. The user can check the reflection of his or her eye in the reflective surface to align the outlet 17 with his or her eye. FIG. 8 shows a view of the rest 30 being positioned on the user's eye surrounding anatomy. In this figure the rest is positioned on the eyebrow of the user. The rest 30 bears against the eyebrow and allows for stability when delivering fluid. FIGS. 7 and 8 show part of the method of use of the apparatus 10 and system 1.

The apparatus 10 or cap and its constituent parts are arranged for use with any existing eyedropper bottle. The apparatus 10 is a retrofittable apparatus that can be easily interchanged between various eyedropper bottles because the apparatus 10 is easily fitted to any bottle due to the shape of the skirt 50, the projection 13 and the threads 70. The apparatus 10 is advantageous because it can be used with a wide range of existing eyedropper bottles or as part of a system for fluid delivery, in conjunction with the container 2 described earlier.

In an alternative embodiment the apparatus 10 and container 2 may be integrally formed together. The apparatus 10 may not be removable from the container 2.

The system 1 for fluid delivery comprises a container 2 and an integral apparatus 10.

The system 1 and container 2 described earlier can also be used for delivery of other fluids besides eye medicaments. Such as for example, ear medicament or nasal medicament or oral medicament. It should be understood that the apparatus 10, system 1 and container 2 will be suitably adapted for use in ear medicament, nasal medicament or oral medicament delivery. For example the apparatus dimensions may be varied to accommodate the apparatus for use in other such related applications.

The apparatus could be used to deliver any fluid, including non-medical fluids such as saline.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A closure apparatus for a device for delivering fluid to an eye, the closure apparatus comprising a lid arranged to be moved from a first position, in which the lid closes a fluid delivery opening of the device, to a second position, in which the fluid delivery opening of the device is open to enable dispensation of fluid, the closure apparatus having an eye rest projecting outwardly from a side wall of the lid that facilitates opening of the fluid delivery opening, the eye rest being arranged to rest against the eye surrounding anatomy of a user when the lid is in the second position during the dispensation of fluid in order to steady the device for delivery of fluid.

2. A closure apparatus in accordance with claim 1 comprising a base that is arranged to engage with a fluid container, the lid of the closure apparatus being arranged to mate with the base to close the fluid delivery opening.

3. (canceled)

4. A closure apparatus in accordance with claim 1 wherein the eye rest bears a message.

5. A closure apparatus in accordance with claim 1 wherein the eye rest comprises a contoured surface that is arranged to conform to the eye surrounding anatomy of a user such that the eye rest bears against the eye surrounding anatomy of a user to allow for greater stability of the apparatus when in use.

6. (canceled)

7. A closure apparatus in accordance with claim 1,wherein the lid is coupled to the base by a hinge, the hinge being disposed generally opposite the eye rest and connecting to a side wall of the base, the hinge is arranged to facilitate movement of the lid from the first position to the second position, and lock the lid in the second position when the lid position exceeds a predefined locking angle.

8. A closure apparatus in accordance with claim 1 comprising a nozzle for the dispensation of fluid from the fluid delivery opening of the device, and a collar disposed concentrically about the nozzle, the nozzle being seated within the collar with the fluid delivery opening disposed below the top of the collar, whereby to protect the nozzle.

9. A closure apparatus in accordance with claim 1 wherein the collar has at least one slot, the at least one slot being arranged to guide fluid out of the collar.

10. A closure apparatus in accordance with claim 8, wherein the collar comprises an upper edge with a projecting lip, the lip being arranged to break the surface tension of any fluid about the collar so as to reduce the chance of fluid flowing back into the nozzle opening.

11. A closure apparatus in accordance with claim 8 comprising a reflective surface disposed about the nozzle, the reflective surface in use enabling a user to view alignment of the nozzle and the fluid delivery opening with their eye and accurately deliver fluid to the desired area.

12-16. (canceled)

17. A closure apparatus in accordance with claim 1 wherein the eye rest is integrally formed with the lid.

18. A closure apparatus in accordance with claim 1 wherein the eye rest extends substantially perpendicular to the side wall of the lid.

19. A closure apparatus in accordance with claim 1 wherein the eye rest functions as a lever to facilitate opening of the fluid delivery opening.

20. An apparatus for facilitating delivery of a fluid comprising a translucent or transparent container arranged to contain the fluid and a closure apparatus in accordance with claim 1.

21. A method of delivering fluid to a user's eye, using an apparatus for facilitating the delivery of fluid, comprising the steps of:
opening a lid of the apparatus, the lid having an eye rest projecting outwardly from a side wall that facilitates opening,
positioning the eye rest on or about eye surrounding anatomy such that the eye rest bears against at least part of the eye surrounding anatomy,
aligning an outlet of the apparatus with the user's eye and providing fluid to the user's eye via the outlet.

22. A method in accordance with claim 21 wherein the step of aligning the outlet comprises the additional step of viewing a reflection of the user's eye within a reflective surface disposed on an upper surface of the apparatus.

23. A method in accordance with claim 21 wherein the apparatus for facilitating the delivery of fluid includes a closure apparatus comprising a device for delivering fluid to an eye, the closure apparatus comprising a lid arranged to be moved from a first position, in which the lid closes a fluid delivery opening of the device, to a second position, in which the fluid delivery opening of the device is open to enable dispensation of fluid, the closure apparatus having an eye rest projecting outwardly from a side wall of the lid that facilitates opening of the fluid delivery opening, the eye rest being arranged to rest against the eye surrounding anatomy of a user when the lid is in the second position during the dispensation of fluid in order to steady the device for delivery of fluid.

24. An eye drop dispenser comprising:
a base that engages with a fluid container, the base having a nozzle that defines a fluid opening for dispensing eye drops from the fluid container,
a lid that mates with the base to close the fluid opening, the lid having a cap that is generally commensurate with the fluid opening of the nozzle,
a hinge that couples the lid to the base so that the cap blocks the fluid opening of the nozzle when the lid is in a closed position, the hinge connecting to a side wall of the base, and
an eye rest that extends outwardly from a side wall of the lid to rest against a user's eye surrounding anatomy during dispensation of eye drops, the eye rest being disposed generally opposite the hinge to facilitate opening of the lid.

25. The dispenser of claim 24 comprising a reflective surface disposed about the nozzle that aids alignment of the nozzle with a user's eye.

26. The dispenser of claim 24 comprising a collar disposed about the nozzle that shields the nozzle from contamination, the fluid opening being disposed below an upper lip of the collar.

27. The dispenser of claim 26 wherein the collar includes a slot that defines an opening for the passage of eye drops.

28-33. (canceled)
